(19) United States
(12) Patent Application Publication
(10) Pub. No.: US 2002/0182227 A1
(43) Pub. Date: Dec. 5, 2002

(54) Treatment of virus using chelator and antiviral agent

(76) Inventor: Bruce Halstead, Grand Terrace, CA (US)

Correspondence Address: Robert D. Fish, Suite 1400, 611 Anton Blvd., Costa Mesa, CA, 92626, US

(21) Appl. No.: 10/159,723

(22) Filed: May 29, 2002

Related U.S. Application Data
(60) Provisional application No. 60/294,481, filed on May 30, 2001.

(51) Int. Cl. C12Q 1/70; G01N 33/53; A61K 39/12

(52) U.S. Cl. 424/204.1; 435/5; 435/7.1

(57) ABSTRACT

A pharmacological composition comprises an antiviral agent and a chelator in a quantity sufficient to reduce a serum concentration of a bivalent metal in an amount of at least 25%, wherein preferred antiviral agents include protease inhibitors, reverse transcriptase inhibitors, and/or an antibody, and wherein preferred chelators chelate at least one of Ca²⁺ and Mg²⁺.

This application claims the benefit of U.S. provisional application No. 60/294,481 filed May 30, 2001, incorporated herein by reference in its entirety.

### FIELD OF THE INVENTION

The field of the invention is antiviral compositions.

### BACKGROUND OF THE INVENTION

Viral infections are unfortunately an almost unavoidable challenge to the health of most human and other mammals, and while many viral infections are successfully cleared by the immune system of the infected individual before substantial damage arises, some viral infections lead to severe damage or even death. There are many known antiviral drugs, however, all or almost all of them suffer from one or more disadvantages, most notably adverse side-effects, built-up of viral resistance, complicated administration schedules, and often high cost. Therefore, there is a need for simple and effective antiviral compositions that are well tolerated, simple to administer, and relatively inexpensive.

### DETAILED DESCRIPTION

The inventors discovered that treatment of a viral infection can be significantly improved by coadministration of an antiviral agent with a chelator. More specifically, the inventors contemplate that particularly suitable chelators deplete the viral environment sufficiently to promote disintegration of the viral envelope.

Consequently, the inventors contemplate a pharmacological composition that includes an antiviral agent and a chelator in a quantity sufficient to reduce a serum concentration of a bivalent metal in an amount of at least 25%.

Suitable antiviral agents particularly include direct antiviral agents and indirect antiviral agents. As used herein, the term “direct antiviral drug” refers to an agent that directly interferes with one or more viral components. For example, virus protein specific antibodies, reverse transcriptase inhibitors or protease inhibitors are considered direct antiviral agents, because such compounds directly bind and to and/or reduce the activity of their respective viral target structures. As also used herein, the term “indirect antiviral drug” refers to a compound that indirectly interferes with a replication or propagation of a virus, and particularly include immunomodulatory agents (e.g., cytokines, various nucleoside analogs, and/or Zn²+). However, it should be appreciated that chelators are explicitly excluded from the definitions of direct and indirect antiviral compounds.

Especially preferred antiviral compounds include plant extracts and/or one or more isolated compounds (isolated from the plant or synthesized de novo) that are present in a plant extract demonstrated to have an antiviral effect. Particularly suitable plants for contemplated extracts and isolated compounds include Abies webbiana; Acacia spec. Acacia Arabia; Agrimonia eupatoria; Ajuga decumbens; Allium cepa; Allium sativum; Aloe vera; Altemanthera philoxeroides or sessiles; Ammi maius; Andographis paniculata; Apium graveolens; Apium leptophyllum; Arachis hypogaea; Arctium lappa; Amebia euhcroma; Asparagus racemosus; Astragalus spinosus; Astragalus lentingosis swainsonine; Buchenavia capita; Bryonia cretica ssp. Dioica; Bryonia angustifolia; Camellia theifera; Camellia sinensis; Cedrela toona; Chrysanthemum morifolium; Coffea arabica; Coptis chinesis; Coptis teetoides; Coptis japonica; Coraria nepalensis; Coriandrum sativum; Curcuma longa; Datura metel syn alba; Daucus carota; Echinacea angustiflora and purpurea; Echinacea simulata; Echinacea pallida; Epimedium grandiflorum; Epimedium sagittatum; Epimedium sinense; Epilobium angustifolium; Erigeron Canadensis; Eugenia or Syzigium claviflorum; Fagara xanthox; Foeniculum vulgarel; Gardenia coronaria; Gaultheria trichophylla; Glycine max; Glycyrrhiza labra; Gossypium herbaceum; Heracleum sphondylium; Hypericum perforatum; Hypericum japonicum; Hyssopus officinalis; Jasminum officinale; Lithospermum erythrorhizon; Lonicera japonica; Luffa luffa; Lycopus europaeus; Magnolia officinalis; Mallotus repandus; Mallotus philippinesis; Matricaria chamomil; Matricaria recutitia; Melissa parviflora; Melissa officinalis; Momordica balsamina; Momordica charantia; Narcissus tazetta; Narcissus pseudonarcissus; Oenthera rosea; Paeonia spec.; Papaver somniferum; Perilla frutescens; Phyllanthus niruri; Pinus koraicenis; Pinus parviflora; Piper nirgum; Plumeria rubra; Polyantha suberosa; Prunella vulgaris; Prunus bakariensis; Prunus amygdalus; Psoralea corylifolia; Randia dunatorum; Raphanus sativus; Rheum palmatum; Rhus coriaria; Rhus chinesis; Ricinus communis; Rosmarinus officinalis; Salvia miltiorhiza and officinalis; Sambucus ebulus; Saussurea lappa; Scilla griffithii; Scutellaria baicalensis baiealein; Sedum sediforme; Senecio scandens; Senecio aereus; Skimmia laureola; Solarium niporum; Swertia franchetiana; Terminalia chebula; Terminalia catappa; Terminalia alata; Thula occidentalis; Trapalaponica spec.; Trichosanthes dioica; Trichosanthes kirilowii; Urtica dioica; Viola yeodensis; Woodfordia fruticosa; Woodwardia spec. Zanoxylum nitidum;

With respect to the chelator it is generally contemplated that all chelating agents are suitable for use in conjunction with the teachings presented herein so long as such chelators (a) reduce serum concentration of a bivalent metal (e.g., Ca²⁺ and Mg²⁺) in an amount of at least 25%, and (b) are at least partially effective to promote viral disintegration at an administered dosage. Particularly contemplated bivalent metals include Ca²⁺ and Mg²⁺. Particularly preferred chelators include 1,2-Bis(2-aminophenoxy)ethane-N,N,N′,N′-tetraacetic acid, Ethylenebis(oxyethylenenitrilo)tetraacetic acid, 1,2-bis(2-aminophenoxy)ethane-N,N,N′,N′-tetraacetic acid tetrakis(acetoxymethyl ester), trans1,2-diaminocyclohexane-tetraacetic acid, and diethyllenetriamine-pentaacetic acid, trimethylaminetricarboxylic acid, poly(aspartic acid), and poly(glutamic acid), ethylenediamine-N,N,N′,N′-tetraacetic acid, and EGTA.

It is further contemplated that suitable compositions will reduce the viral serum titer of a virus in an amount of at least 10% (e.g., as determined by RT-PCR), and especially contemplated viruses include retroviruses (e.g., HIV, HCV), dsDNA and ssDNA viruses.

With respect to the administration of contemplated compositions, it should be recognized that various protocols are suitable, and especially contemplated protocols include substantially simultaneous administration of the chelator (e.g., coadministration in a single tablet), or administration of the chelator (or antiviral agent) while there is a measurable concentration of the antiviral agent (or chelator) in the patient. For example, it is contemplated that suitable antiviral agents may be orally administered, while the chelator is parenterally administered (e.g., via injection or mucosal presentation).

Consequently, the dosage and formulation of contemplated antiviral agents and chelators may vary substantially, however, it is preferred that the antiviral agent is administered in approved and/or known dosages and formulations. Similarly, it is preferred that dosages and formulations of appropriate chelators are identical or similar to those known in the art.

Thus, specific embodiments and applications of antiviral treatments using a chelator and an antiviral agent have been disclosed. It should be apparent, however, to those skilled in the art that many more modifications besides those already described are possible without departing from the inventive concepts herein. The inventive subject matter, therefore, is not to be restricted except in the spirit of the appended contemplated claims. Moreover, in interpreting both the specification and the contemplated claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed is:

1. A pharmacological composition, comprising:
an antiviral agent and a chelator in a quantity sufficient to reduce a serum concentration of a bivalent metal in an amount of at least 25%.

2. The composition of claim 1 wherein the antiviral agent is a direct antiviral agent.

3. The composition of claim 2 wherein direct antiviral agent is selected for the group consisting of a protease inhibitor, a reverse transcriptase inhibitor, and an antibody.

4. The composition of claim 1 wherein the antiviral agent comprises an immunostimulatory compound selected for the group consisting of a cytokine, nucleoside analog, and Zn²⁺.

5. The composition of claim 1 wherein the antiviral agent comprises a plant extract.

6. The composition of claim 1 wherein the antiviral agent comprises an isolated compound t hat is present in a plant extract demonstrated to have an antiviral effect.

7. The composition of claim 6 wherein the isolated compound is synthesized de novo.

8. The composition of claim 1 wherein the chelator chelates at least one of Ca²⁺ and Mg²⁺.

9. The composition of claim 8 wherein the chelator is selected from the group consisting of 1,2-Bis(2-aminophenoxy)ethane-N,N,N′,N′-tetraacetic acid, Ethylenebis(oxyethylenenitrilo)tetraacetic acid, 1,2-bis(2-aminophenoxy)ethane-N,N,N′,N′-tetraacetic acid tetrakis(acetoxymethyl ester), trans-1,2-diaminocyclohexane-tetraacetic acid, and diethyllenetriamine-pentaacetic acid.

10. The composition of claim 8 wherein the chelator is selected from the group consisting of trimethylaminetricarboxylic acid, poly(aspartic acid), and poly(glutamic acid).

11. The composition of claim 8 wherein the chelator is ethylenediamine-N,N,N′,N′-tetraacetic acid.

12. The composition of claim 1 wherein the bivalent metal is at least one of Ca²⁺ and Mg²⁺.

13. The composition of claim 1 wherein the composition reduces a viral serum titer of a virus in an amount of at least 10%.

14. The composition of claim 13 wherein the virus is a retrovirus.

15. The composition of claim 14 wherein the retrovirus is an HIV virus or an HCV virus.

16. The composition of claim 13 wherein the viral serum titer is determined by RT-PCR.

17. The composition of claim 1 wherein the antiviral agent is orally administered and the chelator is parenterally administered.
